(19) United States
(12) Patent Application Publication
(10) Pub. No.: US 2023/0145393 A1
(43) Pub. Date: May 11, 2023

(54) BREAST IMPLANT HAVING A FIXING FASTENER

(71) Applicant: Marie-Christine MISSANA, La Varenne Saint-Hilaire (FR)

(72) Inventor: Marie-Christine MISSANA, La Varenne Saint-Hilaire (FR)

(21) Appl. No.: 17/912,827

(22) PCT Filed: Apr. 18, 2020
(86) PCT No.: PCT/EP2020/060938

§ 371 Date: Sep. 19, 2022

(30) Foreign Application Priority Data
May 5, 2019 (FR) FR1904705

(51) Int. Cl. A61F 2/12

(52) U.S. Cl. CPC A61F 2/12; A61F 2220/0008; A61F 2230/0008; A61F 2230/0019; A61B 2017/0414

(57) ABSTRACT

The present invention relates to a breast implant provided with at least one suture attachment (1), such that said fastener comprises a base (2) having a surface interfacing with the implant, said base extending in height into a protuberance (3) defining a volume, in which a suture through-opening (4) is formed.

### TECHNICAL FIELD

The invention relates to breast implants, in particular those intended for breast augmentation surgery or for reconstructive breast surgery. In a known manner, these implants have a flexible envelope made of biocompatible material of the silicone type, which envelope is filled with a fluid, a liquid of the aqueous type or a gel.

The invention is directed in particular to the implants of the simplest designs, so-called symmetrical implants (round ones or those referred to as anatomical). It is directed also and especially to so-called asymmetrical implants, the contours and curvature of which are closer to the shape of a natural breast than are the symmetrical implants. To be more precise, a symmetrical implant can be positioned equally well to the right or left of the patient's thorax. By contrast, an asymmetrical implant, closer to the natural morphology of a breast, has a shape that differs depending on whether the implant is placed to the right or left, since it is adapted to the shape of the thorax (it is not possible to switch the positioning of a right-side asymmetrical implant with that of a left-side asymmetrical implant).

On the other hand, these asymmetrical implants and those called anatomical symmetrical implants have to be fixed correctly and durably, so as not to risk “turning” once they have been implanted in their surgical pocket, thus becoming ungainly and uncomfortable and leading to a deterioration of the cosmetic result, which may lead to surgical revision. It is therefore important to be able to fix the implants in the desired position during their surgical implantation, while also taking account of the possible slight movements, which are normal, of the implant during the weeks or months following its implantation.

### PRIOR ART

To ensure that the implant remains in the desired position after implantation, without rotation, pivoting, reversal or translation other than the abovementioned slight adjustments just after implantation, a first solution lies in giving a particular texture to the envelope of the implant, said texture having a certain relief and a certain roughness allowing its adhesion to the adjacent tissues and therefore its permanent fixation in the desired position. However, recent studies tend to suggest that these textured implants could pose problems in some cases. There is therefore a trend to move back to implants whose envelope has a smooth outer surface, which therefore raises more acutely the question of durable fixation of the implant in the appropriate position in the surgical pocket.

It has already been proposed to add fixing tongues to the implants, allowing the surgeon to fix the implant by sutures through said tongues, as is described for example in the patent application WO 2004/041128: this document concerns connecting tabs which extend beyond at least part of the contour of the flexible envelope of the implant, are of generally crescent shape and are centered on the median axis of the implant. The implant can also have positioning tongues arranged on the anterior face of the implant so as to form an extra thickness that is perceptible to the touch. The connecting tabs described in said document are not without drawbacks, however: they are indeed suturable, of course, but they can become torn when perforated by the suture needle. In addition, the fact that they are arranged only on the posterior face of the implant makes them inaccessible to the surgeon when performing breast augmentation surgery by the areolar route, the transareolar-periareolar route and the inframammary route. Moreover, their arrangement on a median plane also makes them difficult to access for suturing in the case of breast augmentation surgery by an axillary approach. Finally, the fact that they protrude beyond the contour of the implant can make them palpable/detectable in the lower part, at the level of the inframammary fold, which is not desirable as regards the comfort of the person wearing the implant.

The aim of the invention is therefore to improve the design of the implants, and more particularly to improve the means for fixing said implants by suturing, in particular so that said means are easier for the surgeon to use during placement of the implant, are safer in terms of minimizing any movement of the implant once in place, and are more pleasant for the person wearing the implant, by minimizing any discomfort caused by their presence.

### SUMMARY OF THE INVENTION

The invention relates first of all to a breast implant provided with a fastener for fixing by suturing, such that said fastener comprises a base having a surface interfacing with the implant, said base continuing in height into a protuberance defining a volume in which a suture through-opening is formed.

The invention has thus developed a new type of fastener which is fixed by suturing and which is no longer a simple tongue protruding from the implant: it is a three-dimensional tongue and not, as has been known hitherto, a flat tongue having an opening or allowing a hole to be formed at its end. This design affords many advantages: with a suture opening thus arranged and already made, the surgeon can easily perform suturing through the opening.

Since the opening is formed in a protuberance having a certain height compared to the thickness of a simple tongue, it is not tearable, and it is stronger. Since the opening is situated “above” the base of the tongue and not at one of the ends thereof, such a fastener will be able to be placed much more freely on the implant, and in particular without “overshooting” the contour of the implant, as is set out in detail below, which minimizes any discomfort for the person wearing the implant, which implant can become undetectable/impalpable, in contrast to the tongues currently available.

The fastener according to the invention preferably has an elongate shape oriented along a longitudinal axis X, the protuberance and the through-opening also being elongate along an axis X′ substantially parallel to the longitudinal axis X. The advantage of such a configuration is that the elongate opening makes it possible either to pass a thread through several times, or to pass through only one thread, but while allowing a certain movement of the implant in the surgical pocket: in fact, as has been mentioned above, the implant, once in place, tends to move slightly for a period of a few weeks/months, especially vertically. Having an elongate opening, which is then also oriented vertically on the implant, allows the fastener to follow this normal movement after surgery, without risk of pulling on the envelope of the implant or damaging this envelope.

Preferably, the suture thread passes through only a single suture through-opening of a fastener according to the invention (that is to say it will fasten the implant only by suturing through a single opening of the fastener according to the invention).

The suture thread is thus passed through the through-opening of the fastener in a direction substantially parallel to the junction surface between the base and the implant (parallel is understood as meaning the fact that the direction is parallel to the surface of this base/implant junction, or, in the case of a curved surface, parallel to the projection of this curved surface onto a plane).

Preferably, the length of the fastener is equal to at least twice its width, in particular at least three times its width. This elongation permits a protuberance that is also elongate, and a suture opening as well, with good mechanical strength of the assembly.

Preferably, the cross section of the through-opening along its longitudinal axis X′ is oval, elliptical or rectangular with rounded edges. Indeed, for the shape of the opening, as also for that of the fastener as a whole, preference is given to soft, rounded shapes, which are the most suitable for integration in the surgical pocket in the most natural and pleasant as possible manner in relation to the immediate environment of the implant. The fastener of the invention thus configured is particularly ergonomic.

Also preferably, the elongate through-opening is situated substantially at the mid-height of the protuberance. This is understood to mean that the height of the opening, preferably constant, is centered on the height of the protuberance, which is a mechanically optimal configuration. The through-opening advantageously passes through the protuberance along an axis substantially perpendicular to the longitudinal axis of the base.

Preferably, the fastener has an axial symmetry along at least one longitudinal axis X, preferably along two mutually perpendicular axes X, Y; X, Z, Y and Z being respectively its width and its height. The fact that the fastener is symmetrical is advantageous, since it simplifies its manufacture (with simpler molds for example), and it also simplifies the assembly on the implant if the fastener is attached thereto, in particular by limiting the positioning errors.

Preferably, the interface between the base of the fastener and the implant has a contour that is oval, elliptical or rectangular with rounded edges. This rounded/softened shape makes it easier for the fastener to integrate in the implant.

Preferably, the protuberance has flanks and a top, either flat or rounded, with a surface smaller than the interface between the base of the fastener and the implant. A fastener is therefore obtained which preferably does not have a shape of the parallelepipedal type, but rather a protuberance which will have a profile with a rounded longitudinal and/or transverse cross section. As has been mentioned above, the protuberance thus defines a volume large enough to accommodate the opening but advantageously generally remains rounded in shape, for better integration in the surgical pocket, and for better contact with the adjacent tissues with which the suturing is to be performed.

Preferably, and still in the same vein, the junction between the flanks of the protuberance and the top of the protuberance and/or between the flanks of the protuberance and the base of the fastener is advantageously rounded.

Preferably, the protuberance has flanks which are curved, in particular at least partly convex or concave, preferably comprising a zone of convex shape, adjacent to the base, continued to the top by a convex zone. The point of inflection can be situated, for example, at the middle of or in the vicinity of the height of said protuberance. This avoids any sharp angle both at the level of the fastener itself and between the fastener and the implant.

Advantageously, the fastener has an elongate shape along a length L and is arranged on the implant in such a way that said length L is oriented vertically on said implant. In fact, in this configuration, the suture thread will therefore have a certain latitude, corresponding to the length of the opening, for moving relative to the fastener without risk of causing damage by pulling on the envelope of the implant. This allows the implant to take up its final position after placement, by moving slightly, essentially in a vertical plane, hence the preferred orientation of the fastener and its elongate shape.

According to one embodiment, the fastener is arranged on the posterior face of the implant in such a way as to remain contained within the outer contour of said face. Without extending beyond the posterior face, it is therefore not detectable/palpable, particularly at the lower breast fold.

According to another embodiment, the fastener is arranged on the anterior face of the implant at the maximum or in the vicinity of the maximum projection of said anterior face, in particular in the zone corresponding to the areolar zone, in such a way as to remain contained within the outer contour of said face.

Preferably, the implant according to the invention has fasteners both on its posterior face and on its anterior face, a plurality of fasteners on both faces being recommended in order to ensure a fixation of the implant suitable for all the possible incisions in breast augmentation surgery and in reconstructive breast surgery.

The invention relates to a symmetrical implant, provided with at least 6 fasteners, in particular 8 fasteners, on the posterior face, and with at least 1 fastener, in particular 2 fasteners, on the anterior face, preferably arranged in the areolar zone of said anterior face.

The invention also relates to an asymmetrical implant, which is provided with at least 2 fasteners, in particular 4 fasteners, on the posterior face, and with at least 1 fastener, in particular 2 fasteners, on the anterior face.

The implant according to the invention can comprise fasteners attached to the implant or integral with the implant. The fasteners can be attached to the implant by different techniques, for example by adhesive bonding.

Adhesive bonding in particular is especially appropriate when the material of the envelope of the implant and the material of the fastener are chemically compatible, even identical: this may involve silicone or a silicone derivative. It is also possible to use an overmolding technique or other techniques.

In the first case, the invention also relates to the fasteners themselves.

According to a variant, several fasteners, in particular 2 fasteners, can be rigidly connected by a common base, which is therefore provided either with two protuberances, each having its own opening, or with a common protuberance having two openings side by side.

According to an exemplary embodiment, the fastener can have a length L of at most 30 mm, in particular of at most 12 mm, and in particular of at least 3 mm, in particular of at least 6 mm, a width 1 of at most 20 mm, in particular of at most 8 mm, and in particular of at least 2 mm, and a height h of at most 10 mm, in particular of at most 6 mm, and in particular of at least 2 mm.

Advantageously, the implant according to the invention has a smooth outer texture. It is in fact with such a texture that fixation by suturing is most useful/relevant.

The invention will be described in more detail below with reference to non-limiting exemplary embodiments and to the figures, relating to symmetrical and asymmetrical implants.

### LIST OF FIGURES

FIG. 1 shows a perspective view of an example of a fastener for fixing a breast implant by suturing.

FIG. 2 shows a front view of the fastener from FIG. 1.

FIG. 3 shows a side view of the fastener from FIG. 1.

FIG. 4 shows a top view of the fastener from FIG. 1.

FIG. 5 shows a bottom view of the fastener from FIG. 1.

FIG. 6 shows a cross section through the fastener from FIG. 1.

FIG. 7 shows the anterior face of an asymmetrical breast implant provided with fasteners according to FIGS. 1 to 6.

FIG. 8 shows the posterior face of the asymmetrical breast implant according to FIG. 6 provided with fasteners according to FIGS. 1 to 6.

FIG. 9 shows the profile view of the asymmetrical breast implant of FIGS. 7 and 8, seen in profile.

FIG. 10 shows the anterior face of a symmetrical breast implant provided with fasteners according to FIGS. 1 to 6.

FIG. 11 shows the posterior face of the symmetrical breast implant according to FIG. 10 provided with fasteners according to FIGS. 1 to 6.

FIG. 12 shows the profile view of the symmetrical breast implant of FIGS. 10 and 11, seen in profile.

### DESCRIPTION OF THE EMBODIMENTS

The invention relates to fasteners for breast implants. The implants comprise, in a known manner, a flexible envelope, for example made of silicone, filled with a fluid.

The fasteners according to the invention can be of the same material as the envelope and can be integrated in the envelope. The fasteners can also be made of the same material or other equivalent biocompatible material and can then be attached to the envelope by any technique known for this type of material (in the form of an insert in a mold, for example).

### Examples

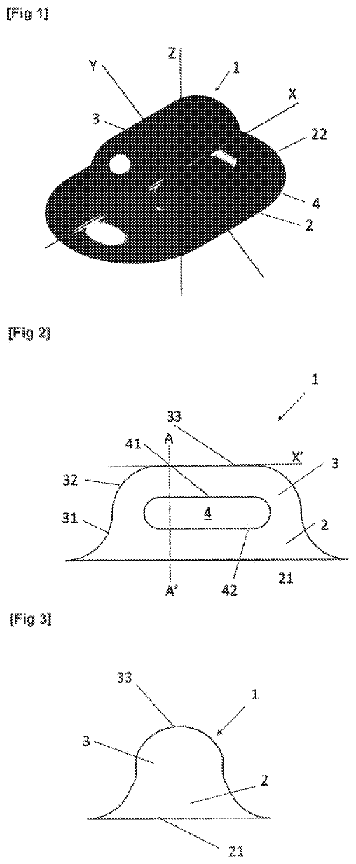

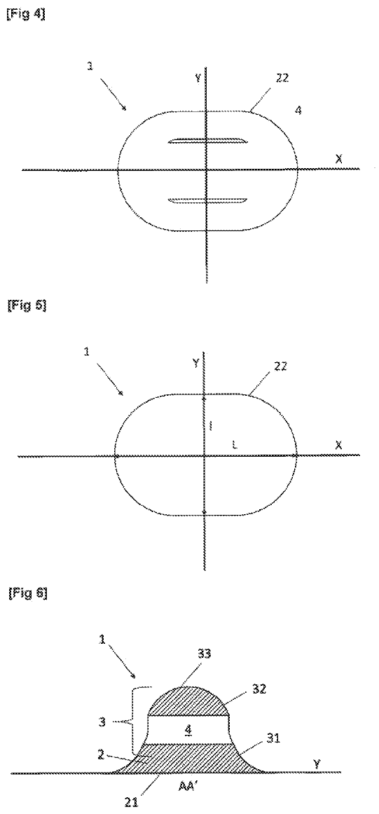

FIGS. 1 to 5 will serve to describe the fastener itself, while FIGS. 6 to 12 will serve to describe the integration/association of the latter with symmetrical or asymmetrical implants.

The fastener 1 shown in FIGS. 1 to 5 (which will be described together) is therefore made of a biocompatible material, for example silicone, and is smooth. It is flexible. In FIGS. 1-5, it is shown placed on a substantially horizontal plane, without other mechanical constraints, and the terms “lower”, “upper”, “bottom”, “top” and other spatial references are understood by considering this fastener according to this convention.

The fastener 1 has an elongate shape. It has a base 2 and a protuberance 3. The base 2 has a lower face 21 intended to be associated/integrated with the breast implant. The contour 22 of the base 2 is here rectangular with rounded edges, but it can also be oval, elliptical, or of shapes similar to these. The base 2 has a longitudinal axis X and a transverse axis Y. The protuberance 3 rises from the base along the vertical axis Z.

It will be noted that the fastener 1 has symmetry with respect to each of these three axes. This is a preferred embodiment of the invention, but it should be noted that fasteners according to the invention do not necessarily have axial symmetries such as these.

The protuberance 3 has flanks with rounded connection to the base, according to both the width and the length of the fastener: with a lower flank portion 31, starting from the base 1, having a convex shape and continuing into an upper flank portion 32 having a concave shape, the inflection point being situated approximately at half the maximum height h of the protuberance measured along the axis Z from the face 21 of the base. The top of the protuberance 3 defines a flat surface 33 with rounded connection to the upper flank portion 32.

Alternatively, the top of the protuberance can also be slightly rounded, but it then preferably has a less pronounced curvature than that of the flanks 32: while it is advantageous for the protuberance to provide a sufficient volume to form a suture opening 4 there, it is also expedient to optimize its volume, for material cost savings and also and especially so that the fastener takes up as little room as possible: a flat or slightly rounded top makes it possible to define a minimum volume in which a suture opening 4 of sufficient dimensions is formed.

This opening 4 therefore passes through the protuberance along an axis substantially perpendicular to the longitudinal axis X of the base. The latter has a longitudinal cross section, along an axis X′ parallel to the axis X, of rectangular shape with rounded edges, although it can equally well be elliptical, oval or of shapes similar to these (FIG. 2). This shape facilitates the passage of the suture needle during the surgical operation, and it then facilitates the positioning/slight movement of the suture thread later, after placement of the implant. The thread is introduced into the opening 4 by the needle in a direction perpendicular to the axis X or obliquely, in a direction between that of the longitudinal axis X and that of the axis Y which is transverse thereto. The opening 4 is wide enough to ensure that this passage perpendicular to X or obliquely is easy and that, after suturing, the thread can move in a direction parallel to the axis Y or obliquely between the axes X and Y.

The opening 4 is centered at the mid-height of the protuberance 3 or in the vicinity of this zone. Along its transverse axis Y′ parallel to the axis Y, it has a substantially rectangular cross section (FIG. 6). It is understood by this that it has an upper face 41 and lower face 42 which are plane and parallel to each other, hence also parallel to the lower face 21 of the base 2 and to the flat top 33 of the protuberance. Laterally, its profile is in continuity with the shape of the flanks of the protuberance 3. Naturally, the longitudinal and transverse cross sections of the suture opening 4 can also have different shapes: thus, in particular, the lower and upper faces may not be flat but possibly at least partly curved.

It will be seen that, like the protuberance 3 and the base 2, the opening 4 is elongate and has the same symmetries, which once again is a preferred embodiment but not essential. It will be seen here that the length of the opening 4 is greater than its width, by a factor of approximately 2, and that its height is at least three times smaller than its length: the height is sufficient for a surgeon to pass a suture needle through it without difficulty. Its length is much greater than the simple passage of a single thread (or several threads) would require: in fact, as is shown in the following figures, this length will offer the necessary clearance, once the implant is in place, for the thread to move a little in the opening until the implant has stabilized/definitively positioned itself in the anatomical pocket after surgical implantation.

The top view of the fastener shows that the shape of the contour of the base and the shape of the top of the protuberance (or its projection on a horizontal plane if it is not absolutely flat) are both very similar, or even identical except for the size, namely rounded. Here again, this is not essential, but it is a preferred configuration.

To give an order of magnitude of the dimensions of the fastener, the following are chosen for example:
a base length of approximately 8 to 10 mm
a base width of approximately 5 to 7 mm
a height (to be understood as the height of the top of the protuberance) of approximately 2 to 4 mm
a suture opening height of 0.3 to 8 mm, and here of approximately 1 to 2 mm or between 0.5 and 1.5 mm
a suture opening length of approximately 2 to 8 mm, in particular 3 to 5 mm (along axis X′)
a suture opening width of approximately 1 to 7 mm, in particular 3 to 5 mm or 2 to 4 mm (along axis Y′).

A ratio r1 length/width of the base of at least 1.5 is recommended, in particular of at least 2, in particular of at least 2.5, and preferably of at most 5.

A ratio r2 length/width of the suture opening 4 of close to or equal to the ratio r1 is recommended.

A ratio r3 of the height h (at the top) of the protuberance 3 to the height of the suture opening 4 of at least 1.25 is recommended, in particular of at least 2, in particular of at most 10.

FIGS. 7 to 9 show the integration of fasteners according to FIGS. 1 to 6 in an asymmetrical implant.

The definitions of the faces of an implant will be recalled firstly:
the so-called “posterior” face of the implant corresponds to the face of the implant intended to be applied to the anterior face of the thoracic cage, when considering the normal position of the breast implant when the latter is in place,
the so-called front or anterior face corresponds to the face of the implant opposite the posterior face.

In all of FIGS. 7 to 12, the implant 10 is shown the way in which it is positioned when implanted, the way it would be spatially arranged when implanted in a person standing with the bust substantially vertical.

The front views of the faces of the implants are represented in the form of four quadrants q1 to q4, which are delimited by dotted lines, this making it possible to distinguish four parts on each face: upper right and left quadrants, lower right and left quadrants.

FIG. 7 is a front view of the anterior or front face fa of an asymmetrical implant, which is provided with two fasteners 1 according to the invention in immediate proximity to the most forward projection point p of the gel-filled or water-filled implant, which constitutes the point of intersection of the four quadrants q1 to q4.

These two fasteners are arranged vertically, that is to say that their longitudinal axis X is oriented vertically. They are arranged in the areolar zone of the anterior face fa, that is to say in a zone of approximately 3 to 4 cm in diameter around the point p, and, in the present case, “straddling” the dotted line separating the upper and lower left quadrants for one of the fasteners, and “straddling” the dotted line separating the upper and lower right quadrants.

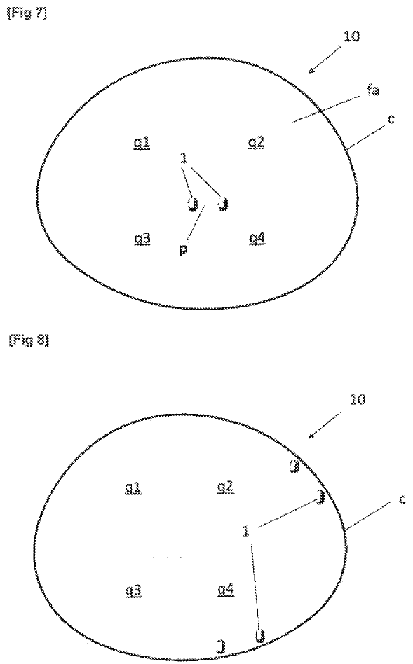

FIG. 8 shows the front view of the posterior face fp of the implant: the posterior face is provided with 4 fasteners, all oriented vertically like the fasteners of the anterior face, and grouped into two pairs of fasteners: two fasteners are arranged in the upper right quadrant, the other two being arranged on the lower right quadrant.

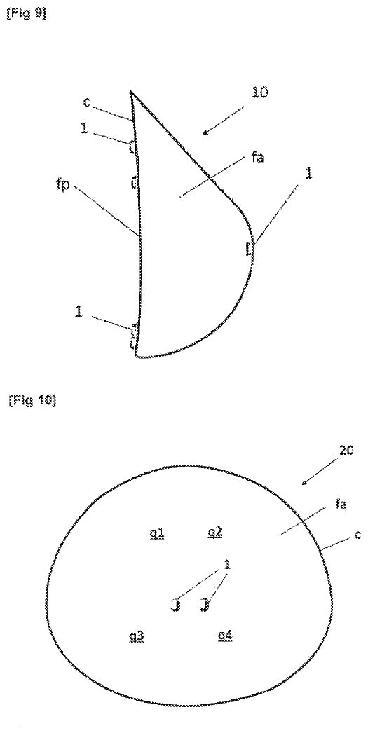

In FIG. 9, the profile view of the implant 10 allows a better understanding of the way in which the 4 fasteners of the posterior face fp are distributed. It can thus be seen that, for each pair of fasteners, the fasteners are offset with respect to each other both in height and laterally, with a less marked difference in height for the pair of lower fasteners than for the pair of upper fasteners. It will also be seen that, for all the fasteners of this implant, nothing “overshoots” the lateral contour c of the implant.

For the positioning of the implant, only two fasteners are necessary and will actually be used by the surgeon:
either he uses the two fasteners on the anterior face according to FIG. 7,
or he chooses two fasteners from among the four fasteners of the posterior face according to FIG. 8.

The choice of the pair of fasteners that will be used in order to ensure the suture depends on the way in which the implant will be put in place, that is to say from the front, which is referred to as implantation by the areolar route or the transareolar-periareolar route, or also by an inframammary route, via the fold situated at the base of the breast, or laterally by the axillary route.

The implant provided with the fasteners according to the invention is therefore extremely versatile and will be able to be implanted by all the possible surgical routes, while remaining “standard” in its design and manufacture.

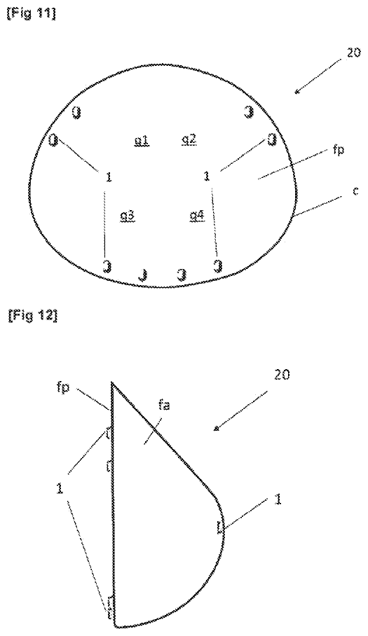

FIGS. 10 to 12 concern the same type of representation, but this time for a symmetrical implant 20. In this case too, all the fasteners are oriented vertically and do not protrude beyond the lateral contour c of the implant.

FIG. 10 shows, in a front view, two fasteners on the anterior face, in the vicinity of the point of maximum projection p, and arranged in the same way as in FIG. 7.

FIG. 11 shows, in a front view, the posterior face of the implant with this time 8 fasteners: a pair of fasteners in the upper left quadrant and, symmetrically with respect to the vertical line separating the right quadrants from the left quadrants, a pair of fasteners in the upper right quadrant, and finally 4 fasteners in the bottom part: a pair in the lower left quadrant and a pair in the lower right quadrant, which are also distributed symmetrically with respect to the aforementioned vertical line. It will be noted that the pairs of fasteners of the upper quadrants are far from the vertical line delimiting the right quadrants from the left quadrants (as in the case of the asymmetrical implant), while the 4 fasteners of the lower quadrants are grouped in the vicinity of this line and are approximately at the same distance from one another.

FIG. 12, showing the symmetrical implant in profile, allows a better understanding of their height distribution, with, as in the case of the asymmetrical implant, more marked height differences for the pairs of fasteners of the upper quadrants compared to the height differences of the fasteners of the lower quadrants. In the same way as for the asymmetrical implant described with the aid of FIGS. 7 and 8, the symmetrical implant requires only two fasteners for its placement, either by the two fasteners on the front face, or by two fasteners on its posterior face; the pair of fasteners that will actually be used to suture the implant is chosen depending on the way in which the implant will be fitted in place surgically.

In conclusion, the fasteners according to the invention are advantageous both in terms of their actual shapes and in terms of their positioning on the implant:
they are elongate and positioned on the implant along a vertical axis in order to leave some “slack”, so as not to pull on the envelope, when the implant is in place, in the month following the intervention under the weight of the prosthesis: they allow the expected movement of the implant after placement to be followed without pain, without any feeling of tightness, while remaining discreet and non-palpable,
their rounded shape does not in any way complicate the placement of the implant which is fitted using them, and it adapts well to adjacent tissues after implantation: they are particularly ergonomic,
they are adapted to all the possible access routes in breast augmentation surgery and also in reconstructive surgery, that is to say the areolar route, the transareolar-periareolar route, the inframammary route, the external mammary route, the axillary route or the mastectomy route: they are particularly versatile and can be standardized in their production,
they are as effective for asymmetrical implants as they are for symmetrical implants, with a recommended 6 fasteners for the former and a recommended 10 fasteners for the latter (only one pair of fasteners being actually used).

The size of the fasteners can vary to a certain extent, depending in particular on the size of the implant. It is possible to start from an average standard size, then define a whole range of fasteners of identical shape but larger or smaller, according to homothetic variations.

It is also possible to use fasteners of different sizes or shapes on the same implant, as long as they comply with the characteristics of the invention.

The techniques for fitting the implant in place using the fasteners according to the invention are described in outline below:
In the case of breast augmentation surgery, the skin incision is dictated by several factors: choice of the operating surgeon, the choice of the patient, whether or not it is necessary to reduce the skin envelope, etc. If a transareolar-periareolar incision or a hemiareolar incision is preferred, the surgical pocket is created, the implant is positioned, and the fasteners are sutured, with the aid of non-absorbable suture thread, to the overlying glandular tissue. If an inframammary incision is preferred, the surgical pocket is created, the implant is positioned, and the fasteners are sutured, with the aid of non-absorbable suture thread, to the underlying muscle tissue or fascia.
If an axillary incision is preferred, the surgical pocket is created, the implant is positioned, and the fasteners are sutured, with the aid of non-absorbable suture thread, to the underlying tissue. In the case of reconstructive breast surgery, the skin incision is dictated by several factors: reconstruction performed at the same time as the removal of the breast or at a later stage, reconstruction by a prosthesis alone or by a prosthesis coupled with mobilization of the patient's tissue or “flap”, choice of the operating surgeon, the choice of the patient, nicotine poisoning, history of radiotherapy, etc.

The prosthesis is easily fixed by the anterior, external or lower fasteners sutured with the aid of non-absorbable suture thread to the surrounding tissue.

1-20. (canceled)

21. A breast implant (10; 20) provided with at least one fastener (1) for fixing by suturing, said fastener (1) comprising:
a base (2) having a surface interfacing with the breast implant, characterized in that said base continues in height into a protuberance (3) defining a volume in which a suture through-opening (4) is formed.

22. The breast implant (10; 20), as claimed in claim 21, wherein:
the fastener (1) has an elongate shape oriented along a longitudinal axis (X), the protuberance and a cross section of the through-opening (4) also being elongate along an axis (X′) substantially parallel to the longitudinal axis (X).

23. The breast implant (10; 20), as claimed in claim 22, wherein:
the cross section of the through-opening (4) along its longitudinal axis (X′) is oval, elliptical or rectangular with rounded edges.

24. The breast implant (10; 20), as claimed in claim 23, wherein:
the length of the fastener (1) is equal to at least twice its width, in particular at least three times its width.

25. The breast implant (10; 20), as claimed in claim 21, wherein:
the through-opening (4) is elongate and is situated substantially at the mid-height of the protuberance (3).

26. The breast implant (10; 20), as claimed in claim 21, wherein:
the through-opening (4) passes through the protuberance along an axis (Y) substantially perpendicular to the longitudinal axis (X).

27. The breast implant (10; 20), as claimed in claim 21, wherein:
the fastener (1) has an axial symmetry along at least one longitudinal axis (X), preferably along two mutually perpendicular axes (X, Y; X, Z).

28. The breast implant (10; 20), as claimed in claim 21, wherein:
the interface between the base (2) of the fastener (1) and the implant has a contour (22) that is oval, elliptical or rectangular with rounded edges.

29. The breast implant (10; 20), as claimed in claim 21, wherein:
the protuberance (3) has flanks (31, 32) and a top (33), either flat or rounded, with a surface smaller than the interface (21) between the base (2) of the fastener (1) and the implant.

30. The breast implant (10; 20), as claimed in claim 29, wherein:
a junction between the flanks (32) of the protuberance and the top (33) of the protuberance (3) and/or between the flanks (32) of the protuberance (3) and the base (2) of the fastener is rounded.

31. The breast implant (10; 20), as claimed in claim 21, wherein:
the protuberance (3) has flanks (31, 32) which are curved, in particular at least partly convex or concave, preferably comprising a zone of convex shape, adjacent to the base, continued to the top (33) by a convex zone.

32. The breast implant (10; 20), as claimed in claim 22, wherein:
said fastener (1) has an elongate shape along a length (L) and is arranged on the implant in such a way that said length (L) is oriented vertically on said implant.

33. The breast implant (10; 20), as claimed in claim 32, wherein:
the fastener (1) is arranged on the posterior face (fp) of the implant in such a way as to remain contained within the outer contour (c) of said face.

34. The breast implant (10; 20), as claimed in claim 21, wherein:
the fastener (1) is arranged on the anterior face (fa) of the implant at the maximum or in the vicinity of the maximum projection (p) of said anterior face, in particular in the zone corresponding to the areolar zone, in such a way as to remain contained within the outer contour (c) of said face.

35. The breast implant (20), as claimed in claim 21, wherein:
the breast implant (20) is symmetrical and is provided with at least six fasteners (1) on the posterior face, and with at least one fastener on the anterior face, preferably arranged in the areolar zone of said anterior face (fa).

36. The breast implant (10), as claimed in claim 21, wherein:
the breast implant (10) is asymmetrical and is provided with at least two fasteners (1), on the posterior face (fp), and with at least one fastener (1), on the anterior face (fa).

37. The breast implant (10; 20), as claimed in claim 21, wherein:
the breast implant (10, 20) comprises a plurality of fasteners (1) attached to the implant or integral with the implant.

38. The breast implant (10; 20), as claimed in claim 37, wherein:
at least two of said plurality of fasteners (1) are rigidly connected by a common base.

39. The breast implant (10; 20), as claimed in claim 21, wherein:
the fastener has a length L of at most 12 mm, a width 1 of at most 8 mm, and a height h of at most 10 mm.

40. The breast implant (10; 20), as claimed in claim 21, wherein:
the breast implant (10, 20) has a smooth outer texture.
